(19) United States
(12) Patent Application Publication
(10) Pub. No.: US 2024/0189596 A1
(43) Pub. Date: Jun. 13, 2024

(54) MEDICAL STIMULATOR AND METHOD OF STIMULATION

(71) Applicant: The Alfred E. Mann Foundation for Scientific Research, Valencia, CA (US)

(72) Inventors: Brian Vernon Mech, Buffalo, MN (US); Neil Talbot, La Crescenta, CA (US); Robert Greenberg, Los Angeles, CA (US); Brian R. Dearden, Pasadena, CA (US); Brian Shelton, Ventura, CA (US); Valma Klein, Canyon Country, CA (US); Sahar Elyahoodayan, Los Angeles, CA (US); Joseph Calderon, Santa Clarita, CA (US); William Brandt, Castaic, CA (US)

(21) Appl. No.: 18/522,030

(22) Filed: Nov. 28, 2023

Related U.S. Application Data
(60) Provisional application No. 63/386,853, filed on Dec. 9, 2022.

(51) Int. Cl. A61N 1/36

(52) U.S. Cl. CPC A61N 1/36053; A61N 1/3606; A61N 1/36139

(57) ABSTRACT

A medical device includes: one or more stimulation leads configured to stimulate at least a person's vagus nerve to treat, via the stimulation of the person's vagus nerve, both obesity and one or more medical conditions comorbid with the obesity; and a controller coupled to the one or more stimulation leads and configured to control and drive the stimulation provided by the one or more stimulation leads.

### CROSS-REFERENCE TO RELATED APPLICATION

This application claims priority to, and the benefit of, Provisional Application No. 63/386,853, filed on Dec. 9, 2022, the entire content of which is incorporated herein by reference.

### BACKGROUND

### 1. Field

The present disclosure relates to utilization of stimulation to treat two or more medical conditions.

### 2. Description of the Related Art

There is a need or desire for continued development of medical devices configured to treat medical conditions such as, for example, obesity, diabetes, obstructive sleep apnea, and/or one or more comorbidities thereof, and methods for treating such medical conditions that provide improved treatment results.

### SUMMARY

Non-limiting aspects of the present disclosure are directed toward utilization of stimulation to treat two or more medical conditions.

Additional aspects will be set forth in part in the description which follows and, in part, will be apparent from the description, or may be learned by practice of the presented embodiments of the disclosure.

According to some non-limiting embodiments, a medical device includes: one or more stimulation leads configured to stimulate at least a person's vagus nerve to treat, via the stimulation of the person's vagus nerve, both obesity and one or more medical conditions comorbid with the obesity; and a controller coupled to the one or more stimulation leads and configured to control and drive the stimulation provided by the one or more stimulation leads.

According to some non-limiting embodiments, a method of treating obesity in a person, the method includes: selecting one configuration of electrodes in a medical device from among at least the following two selectable configurations of electrodes: a first configuration of electrodes wherein a cathode electrode is more proximal to a head of the person than an anode electrode is to the person's head, and a second configuration of electrodes wherein a cathode electrode is more distal to the person's head than an anode electrode is to the person's head; and providing electric stimulation to a vagus nerve of the person utilizing the selected one configuration of electrodes.

### BRIEF DESCRIPTION OF THE DRAWINGS

The drawings, together with the specification, illustrate example embodiments of the present disclosure.

FIG. 1A illustrates a medical system according to some embodiments.

FIG. 1B illustrates a medical device of the system of FIG. 1A according to some embodiments.

FIGS. 2-5 illustrate medical devices according to some embodiments.

FIG. 6 illustrates a medical system, including a medical device illustrated to be implanted in a person, according to some embodiments.

FIGS. 7-12 illustrate medical devices according to some embodiments and implanted in a person.

FIG. 13 illustrates two medical devices according to some embodiments and implanted in a person.

FIG. 14 illustrates a medical device according to some embodiments and implanted in a person.

### DETAILED DESCRIPTION

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section, without departing from the spirit and scope of the present disclosure.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises,” “comprising,” “includes,” and “including,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of”, “at least one selected from”, etc., when preceding a list of elements, modify the entire list of elements and do not modify the individual elements of the list. For example, “at least one of a, b or c”, “at least one selected from a, b and c”, etc., may indicate only a, only b, only c, both (e.g., simultaneously) a and b, both (e.g., simultaneously) a and c, both (e.g., simultaneously) b and c, all of a, b, and c, or variations thereof.

Further, the use of “may” when describing embodiments of the present disclosure refers to “one or more embodiments of the present disclosure.”

Furthermore, any element in a claim that does not explicitly state “means for” performing a specified function, or “step for” performing a specific function, is not to be interpreted as a “means” or “step” clause as specified in 35 U.S.C. § 112(f). In particular, the use of “step of” or “act of” in the claims herein is not intended to invoke the provisions of 35 U.S.C. § 112(f).

All references herein to the “invention”, “our system”, “proposed system”, and the like shall mean (or refer to) embodiments of the invention.

It will be understood that when an element is referred to as being “on”, “connected to”, “coupled to”, or “adjacent to” another element, it can be directly on, connected to, coupled to, or adjacent to the other element, or one or more intervening elements may be present. In contrast, when an element is referred to as being “directly on,” “directly connected to,” “directly coupled to,” or “immediately adjacent to” another element, there are no intervening elements present.

As used herein, the term “substantially,” “about,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

Non-limiting and non-exhaustive embodiments of medical devices, medical systems including a medical device, and methods of providing stimulation treatment will now be described herein with reference to the drawings.

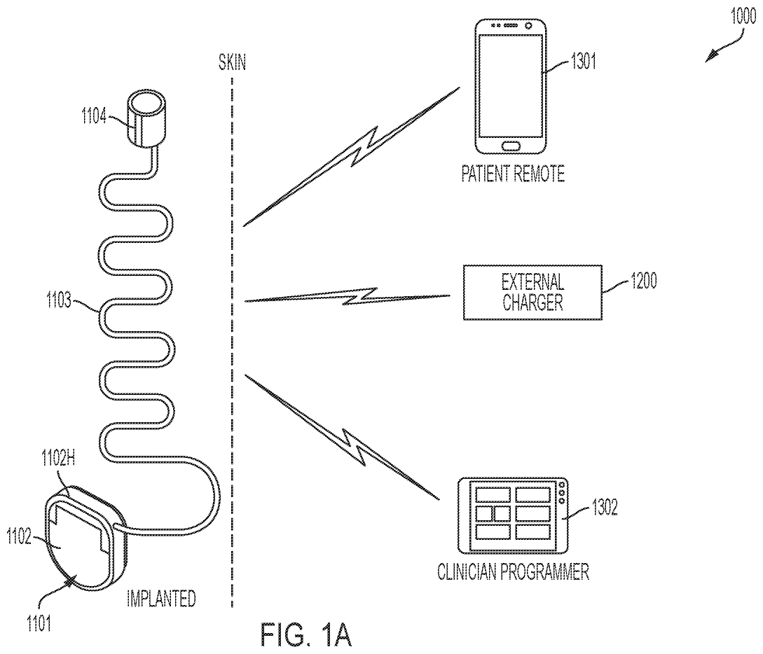

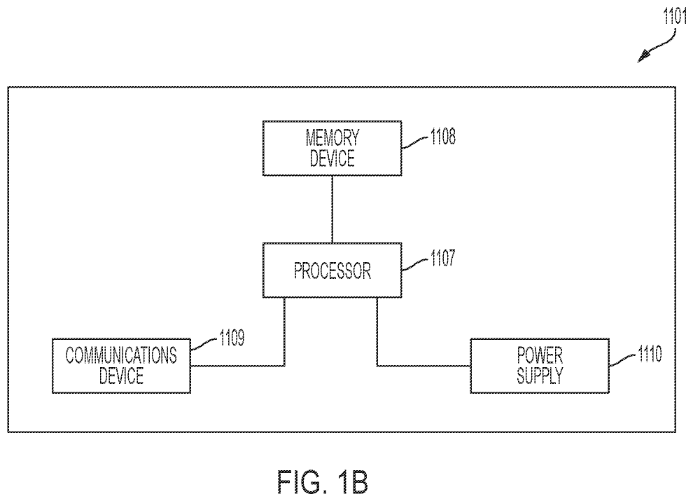

FIG. 1A illustrates a medical system 1000 according to some embodiments. FIG. 1B illustrates a medical device 1101 of the medical system 1000 of FIG. 1A according to some embodiments.

Referring to FIG. 1A, the medical system 1000 may include a medical device 1101 configured to treat one or more medical conditions. The medical device 1101 may be an at least partially implantable medical device configured to provide stimulation to biological tissue in proximity with the medical device 1101 and/or an at least partially externally operable medical device configured to provide stimulation to biological tissue of a person from an outside of the person (e.g., through the person's skin). For example, the medical device 1101 may be an implantable pulse generator configured to provide electrical stimulation through one or more electrodes to treat one or more medical conditions.

In some embodiments, the medical device 1101 is configured to treat obesity or to treat both obesity and one or more medical conditions comorbid with obesity. As used herein, “obesity” may refer to any condition satisfying one or more medical conditions of overweightness (e.g., a BMI of 25 or greater and less than 30), class 1 obesity (e.g., a BMI of 30 or greater and less than 35), class 2 obesity (e.g., a BMI of greater than 35 and less than 40), or class 3 obesity (e.g., a BMI of 40 or greater). The one or more conditions comorbid with obesity may include epilepsy, sleep apnea, obstructive sleep apnea (OSA), central sleep apnea (CSA), diabetes, hypertension, heart failure, depression, gastroesophageal reflux disease, hypercholesterolemia, pelvic floor disorders, chronic inflammation, auto-immune diseases, and/or asthma.

Conditions comorbid with obesity can be caused by, or aggravated by, the obesity. Accordingly, treating both a condition comorbid with obesity as well as the obesity itself, can improve a person's health and can yield treatment results superior to treating the comorbid condition by itself. Furthermore, in some embodiments, both obesity and conditions comorbid with obesity can be treated using stimulation treatment. For example, obesity can be treated by stimulating the vagus nerve (e.g., a branch of the vagus nerve), and conditions comorbid with obesity can be treated by stimulating one or more of the vagus nerve (e.g., a same or different portion of the vagus nerve as is stimulated to treat obesity), the heart, the baroreceptor, the renal nerve, the splenic nerve, the carotid sinus nerve, the parenchyma tissue, the pancreas, the pancreatic nerve, the spinal cord, the hypoglossal nerve, the ansa cervicalis, the pharyngeal nerve, the cranial nerve, the nerve innervating the live, and the phrenic nerve. Thus, according to some embodiments, a medical device configured to provide stimulation to treat both a condition(s) comorbid with obesity and the obesity can not only provide superior treatment results compared to treating only the comorbid condition(s), but can also allow treatment of the obesity with only minor additional invasion (e.g., tunneling of electrical leads of an implantable stimulator) of the body beyond what is needed to treat the comorbid condition(s).

The medical device 1101 (e.g., a medical stimulator) may include a housing 1102 (e.g., a case) including a header 1102H and one or more leads 1103 (e.g., stimulation leads, for example, electrical leads) coupled (e.g., electrically coupled) to the housing 1102 (e.g., to the header 1102H). In the depicted embodiment, the medical device 1101 includes one electrical lead 1103. In some other embodiments, the medical device 1101 may have more than one lead, as discussed in more detail below. In some embodiments, the medical system 1000 may include any medical device within the scope of the present disclosure, including any medical device described and/or illustrated herein or in the attached appendix.

Each of the one or more leads 1103 may include or be electrically coupled to one or more stimulators and/or one or more sensors. The stimulators may be configured to provide stimulation, including at least one of electrical stimulation, inductive stimulation, mechanical stimulation, acoustic stimulation, magnetically induced stimulation, or optical stimulation. For example, each electrical lead may include at least one of one or more electrodes, one or more conductors (e.g., conductor coils configured to generate magnetic fields in response to being driven with alternating current), one or more von Frey filaments, one or more piezoelectric devices, one or more light-emitting diodes, or one or more lasers.

The sensors may be configured to sense (e.g., detect and/or measure) one or more biomarkers, one or more bioindicators, and/or one or more biophysical properties. The one or more biomarkers, one or more bioindicators, and/or one or more biophysical properties may include information relating to the one or more medical conditions (e.g., the status of, or change in, the one or more medical conditions) to be or being treated. For example, the one or more biomarkers, one or more bioindicators, and/or the one or more biophysical properties may include information relating to a metabolism (e.g., metabolic rate) and/or nervous activity relating to hunger/satiety of the person to be treated or being treated. In some embodiments, the one or more biomarkers, one or more bioindicators, and/or the one or more biophysical properties may include information relating to diabetes in the person, for example, information relating to glucose (e.g., glucose levels and/or glucose production), HbA1c (e.g., HbA1c levels and/or HbA1c production), H1A1c (e.g., H1A1c levels and/or H1A1c production), insulin (e.g., insulin levels, insulin production, and/or insulin sensitivity), GLP-1 (e.g., GLP-1 levels and/or GLP-1 production), glucagon (e.g., glucagon levels and/or glucagon production), tumor necrosis factor (TNF) (e.g., TNF levels and/or TNF production), heart rate, heart rate variability, blood pressure, and/or nerve sensors whose signals could provide information relating to glycemic levels. In some embodiments, the sensor may be configured to sense information relating to respiration effort of the person being treated, which may be useful for determining when to provide stimulation to a hypoglossal nerve (HGN). In some embodiments, at least some of one or more sensors are implantable, electrically coupled to the housing 1102, and communicatively coupled to a controller or processor 1107 (see FIG. 1B). In some embodiments, at least some of one or more sensors are operable from the exterior of the person to be treated or being treated and may be communicatively coupled (e.g., wirelessly communicatively coupled) to the controller or processor 1107. In the context of the present application and as an example, the controller or processor 1107 may include or be a microcontroller or microprocessor.

Although in the depicted embodiment the electrical lead 1103 includes an electrode 1104 for providing stimulation, and other embodiments illustrated herein depict medical devices including one or more electrodes for providing stimulation and/or one or more electrodes for sensors, it will be understood that the present disclosure is not limited thereto, but rather, includes embodiments where one or more other types of stimulators and sensors are used instead of, or in addition to, the electrode(s) described or illustrated in the depicted embodiments.

The housing 1102 may be configured to contain at least some components of the medical device 1101 (as described in more detail below) and substantially electrically insulate the components contained within the housing 1102, for example, from surrounding biological tissue. The housing 1102 may have one or more openings (e.g., in the header 1102H) configured to receive at least part of the one or more electrical leads 1103.

The medical system 1000 may include an external charger 1200 configured to wirelessly charge or power (e.g., via induction) the medical device 1101. For example, the medical device 1101 may be configured such that the medical device 1101 can store energy wirelessly received from the external charger 1200 in a rechargeable battery and/or the medical device 1101 may be configured such that the medical device 1101 can use energy received from the external charger 1200 for at least some of the operations (e.g., providing stimulation and/or providing power to at least some of the components) of the medical device 1101 without first storing the energy in a rechargeable battery.

The medical system 1000 may further include one or more controller devices configured to control (e.g., wirelessly control) at least some of the operations of the medical device 1101. For example, the controller device(s) may be communicatively coupled (e.g., wirelessly communicatively coupled via Bluetooth™, Bluetooth Low Energy, etc.) to the medical device 1101. In the depicted embodiment, the medical system 1000 includes a clinician programmer device (e.g., programmer) 1302 and a patient remote 1301 configured to control at least some of the operations of the IPG 101.

Referring to FIG. 1B, the medical device 1101 may include the processor 1107; a memory device 1108, for example, a non-volatile memory device (e.g., flash memory, or read-only memory (ROM), such as programmable read-only memory (PROM) or erasable programmable read-only memory (EPROM)); a communications device 1109 (e.g., a receiver, a transmitter, and/or a transceiver); and/or a power supply 1110 (e.g., a primary battery and/or a rechargeable battery). The communications device 1109 may be configured to provide wireless communication links (e.g., through the skin of a person in whom the medical device 1101 may be implanted) to the clinician programmer device 1302 and/or to the Patient remote 1301. Wireless links may include Bluetooth, Bluetooth Low Energy, or other protocols with suitable authentication and encryption to protect patient data. In some embodiments, the memory device 1108, the communications device 1109, and/or the power supply 1110 are communicatively coupled to each other over processor 1107. The processor 1107, the memory device 1108, the communications device 1109, and/or the power supply 1110 may be contained in the housing 1102, and the proximal ends of the one or more electrical leads 1103 may be electrically coupled to the power supply 1110. In some embodiments, the IPG 101 includes additional components, such as a timer (e.g., a real-time clock (RTC)) configured to measure the passage of time, one or more drivers configured to drive the one or more stimulators (e.g., to provide an electric current to an electrode and/or to a conductor coil), and/or a controller (e.g., a microcontroller and/or a microprocessor) coupled to the one or more drivers and configured to control the one or more drivers' driving of the one or more stimulators. In some embodiments, the controller may be included in the processor 1107. For each of the operations, processes, and functions of the medical device 1101 disclosed herein (e.g., operations, processes, and functions that the medical device is configured to perform or provide), the memory device 1108 may store computer readable instructions that, when executed by the controller or the processor 1107, cause the medical device 1101 to perform such operations.

As used herein, the terms “processor” (e.g., processing circuits) may include any combination of hardware, firmware, memory and software, employed to process data or digital signals. The hardware of a controller may include, for example, a microcontroller, a microprocessor, application specific integrated circuits (ASICs), general purpose or special purpose central processors (CPUs), digital signal processors (DSPs), graphics processors (GPUs), and programmable logic devices such as field programmable gate arrays (FPGAs). In a processor, as utilized herein, each function is performed either by hardware configured (e.g., hard-wired) to perform that function, or by more general purpose hardware, such as a CPU, configured to execute instructions stored in a non-transitory storage medium or memory. A processor may contain two or more processors, for example, a processor may include two processors, an FPG and a CPU, interconnected on a PCB.

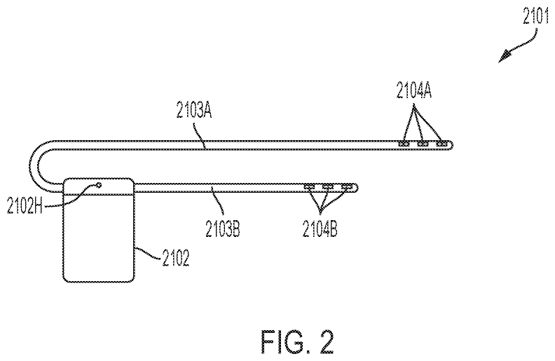

FIGS. 2-5 illustrate medical devices according to some embodiments. In some embodiments, a medical device may be configured to provide first stimulation to treat at least a first medical condition (e.g., obesity) and to provide second stimulation to treat one or more other medical conditions (e.g., one or more medical conditions comorbid with the first medical condition, such as one or more medical conditions comorbid with obesity). In some embodiments, a medical device may be configured to provide at least first stimulation and second stimulation to treat a same medical condition (e.g., to treat obesity, diabetes, or OSA).

The first stimulation may be provided in accordance with a first treatment protocol, and the second stimulation may be provided in accordance with a second treatment protocol. The medical device may be configured (e.g., in response to a controller (e.g., microcontroller and/or microprocessor) or processor of the medical device executing computer-readable instructions stored in a memory device of the medical device) to controllably provide the first stimulation and the second stimulation in accordance with the first and second treatment protocols, respectively. Each of the first and second treatment protocols may include electric current frequency, electric current amplitude, electric current pulse width, electric current duty cycle, electrode configuration (e.g., electrode polarity), a time duration for providing stimulation, and/or a time for an onset of providing stimulation. The first treatment protocol may be configured, when implemented via the first stimulation, to treat a first medical condition, and the second treatment protocol may be configured, when implemented via the second stimulation, to treat one or more other medical conditions (e.g., one or more medical conditions comorbid with the first medical condition). The second treatment protocol may be different from, or substantially the same as, the first treatment protocol. In some embodiments, the first and second treatment protocols may be configured, when respectively implemented via the first and second stimulation, to treat a same medical condition.

A medical device may be configured to provide the first stimulation and the second stimulation at different, or substantially the same, place. For example, the medical device may include one or more first stimulators configured to provide the first stimulation to a first region of a first organ (e.g., a first region of the vagus nerve), and the medical device may include one or more second stimulators configured to provide the second stimulation to provide the second stimulation to a second region of the first organ (e.g., a second region of the vagus nerve spaced apart from the first region on the vagus nerve) or to provide the second stimulation to a second organ (e.g., the heart, the baroreceptor, the renal nerve, the splenic nerve, the carotid sinus nerve, the parenchyma tissue, the pancreas, the pancreatic nerve, the spinal cord, the hypoglossal nerve, the ansa cervicalis, the pharyngeal nerve, the cranial nerve, the nerve innervating the live, and/or the phrenic nerve). In some embodiments, the medical device may be configured to provide the first stimulation and the second stimulation to substantially a same place (e.g., at substantially a same region of the vagus nerve), and the first and second stimulations may be provided during time periods that at least partially do not overlap (e.g., during entirely separate time periods) respectively in accordance with the first and second treatment protocols.

FIG. 2 illustrates a medical device 2101 according to some embodiments. The medical device 2101 may include some features that are similar to, or the same as, features of other medical devices within the scope of the present disclosure, for example, of the medical devices described hereinabove and of medical devices described hereinbelow.

Referring to FIG. 2, the medical device 2101 includes a housing 2102 including a header 2102H; a first lead 2103A (e.g., electrical lead) including one or more first electrodes 2104A and coupled (e.g., electrically coupled) to the housing 2102; and a second lead 2103B (e.g., electrical lead) including one or more second electrodes 2104B and coupled (e.g., electrically coupled) to the housing 2102.

The medical device 2101 may be configured to provide first stimulation to a first portion of the vagus nerve to treat obesity through the one or more first electrodes 2104A and to provide second stimulation to either a second portion of the vagus nerve or to another biological organ (e.g., the heart, the baroreceptor, the renal nerve, the splenic nerve, the carotid sinus nerve, the parenchyma tissue, the pancreas, the pancreatic nerve, the spinal cord, the hypoglossal nerve, the ansa cervicalis, the pharyngeal nerve, the cranial nerve, the nerve innervating the live, and/or the phrenic nerve) to treat one or more medical conditions comorbid with obesity through the one or more second electrodes 2104B.

In some embodiments, the first and second electrical leads 2103A and 2103B may be respectively shaped and sized, and/or the one or more first electrodes 2104A and the one or more second electrodes 2104B may be respectively positioned on the first and second electrical leads 2103A and 2103B, such that the one or more first electrodes 2104A are positionable proximal (e.g., at or near) to the first portion of the vagus nerve and such that the second electrodes 2104B are positionable proximal to the second portion of the vagus nerve or to the second organ. In some embodiments, one selected from the one or more first electrodes 2104A and the one or more second electrodes 2104B are positionable farther from housing 2102 than another one selected from the one or more first electrodes 2104A and the one or more second electrodes 2104B. In some embodiments, the first and second electrical leads 2103A and 2103B may be different in size (e.g., length) and/or the one or more first electrodes 2104A may be positioned at a different distance along the first electrical lead 2103A from the housing 2102 than a distance along the second electrical lead 2103B at which the one or more second electrodes 2104B are positioned.

For example, in the depicted embodiment, the first electrical lead 2103A is longer than the second electrical lead 2103B, and the one or more first electrodes 2104A are positioned at a distance along the first electrical lead 2103A that is greater than a distance along the second electrical lead 2103B at which the one or more second electrodes 2104B are positioned. In some embodiments, the first electrical lead 2103A is shorter than the second electrical lead 2103B, and the one or more first electrodes 2104A are positioned at a distance along the first electrical lead 2103A that is smaller than a distance along the second electrical lead 2103B at which the one or more second electrodes 2104B are positioned. In some embodiments, the first electrical lead 2103A is substantially the same in length as the second electrical lead 2103B, and the one or more first electrodes 2104A are positioned at a distance along the first electrical lead 2103A that is greater or smaller than a distance along the second electrical lead 2103B at which the one or more second electrodes 2104B are positioned.

The medical device 2101 may include one or more first stimulators configured to stimulate a first portion of the vagus nerve to treat obesity, and one or more second stimulators configured for stimulating either the second portion of the vagus nerve or a second biological organ to treat the one or more medical conditions comorbid with obesity. In the depicted embodiment, the one or more first stimulators include the one or more first electrodes 2104A, and the one or more second stimulators include the one or more second electrodes 2104B. The one or more first electrodes 2104A may be configured (e.g., have a number, arrangement, shape, size, rigidity, flexibility, and/or configuration (e.g., polarity)) for stimulating the first portion of the vagus nerve to treat the obesity, and the second electrodes 2104B may be configured (e.g., have a number, arrangement, shape, size, rigidity, and/or configuration (e.g., polarity)) for stimulating the second portion of the vagus nerve or the second biological organ to treat the one or more comorbid conditions. A configuration of electrode(s) may be based at least in part on the shape, size, and/or type of biological organ (or portion thereof) that is to be stimulated and/or on the type of medical condition to be treated. For example, a configuration of electrodes may be helical, having a series of open rings coupled to a common backbone and shaped and sized to at least partially wrap around an elongated nerve. In some other embodiments, a configuration of electrodes may include one or more electrodes that are rigid or flexible and/or that are exposed, protruding, or recessed from the electrical lead. In some embodiments, a configuration of electrodes may include one or more cuff electrodes.

In the depicted embodiment, the one or more first stimulators include three first electrodes 2104A, and the one or more second stimulators include three second electrodes 2104B. In some other embodiments, the one or more first stimulators may include one electrode, two electrodes, or four or more electrodes (e.g., four electrodes, six electrodes, eight electrodes, etc.), and the one or more second stimulators may include one electrode, two electrodes, or four or more electrodes (e.g., four electrodes, six electrodes, eight electrodes, etc.).

In some embodiments, the medical device 2101 is configured to control the polarity of the one or more first electrodes 2104A and/or the polarity of the one or more second electrodes 2104B. For example, the medical device 2101 may be configured to controllably set each electrode of the one or more first electrodes 2104A to be a cathode, to be an anode, or to float (e.g., to not be driven and/or provided with a voltage), and the medical device 2101 may be configured to controllably change the polarity of each electrode.

The one or more first electrodes 2104A may include an alpha electrode and a beta electrode, and the medical device 2101 may be configured to controllably set, and/or to controllably change, the polarity of each of the alpha electrode and the beta electrode. For example, the medical device 2101 may be configured, in response to a controller (e.g., microcontroller and/or microprocessor) or processor of the medical device 2101 executing computer-readable instructions stored in a memory device of the medical device 2101, to controllably set the alpha electrode to be a cathode, to be an anode, or to float and to controllably set the beta electrode to be a cathode, to be an anode, or to float, for example, by controlling one or more drivers configured to drive the first and beta electrodes.

In some embodiments, the medical device 2101 is configured to set the alpha electrode and the beta electrode to have opposite polarities and to controllably reverse the polarities of the alpha electrode and the beta electrode. For example, the medical device 2101 may be configured to respectively set the alpha electrode and the beta electrode to be a cathode and an anode, and to change the polarities of the alpha electrode and the beta electrode so that the alpha electrode is an anode and the beta electrode is a cathode. In some other embodiments, the alpha electrode and the beta electrode can be controllably set to have a same polarity. For example, the one or more first electrodes 2104A may include at least three electrodes including the alpha electrode and the beta electrode, and the medical device 2101 may be configured to controllably set the alpha electrode and the beta electrode to have a same polarity and to be opposite in polarity compared to another electrode of the at least three electrodes (e.g., an electrode between the alpha electrode and the beta electrode).

In some embodiments, the alpha electrode and the beta electrode may be positionable along the vagus nerve of a person, and the alpha electrode may be positionable farther away from a head of the person along the vagus nerve compared to the beta electrode. For example, the alpha electrode and the beta electrode may be positioned at different positions along a length of the first electrical lead 2103A. Because the polarities of the alpha electrode and the beta electrode can be controllably set and changed between at least cathode and anode, the medical device 2101 may be configured to control a position of an anode relative to a cathode along the vagus nerve. When the vagus nerve of a person is stimulated with the anode electrode more proximal to the head of the person than the cathode, the person may experience weight loss at a faster rate than if the anode electrode is more distal to the head than the cathode. In the electrode configuration where the anode is more proximal to the head than the cathode is to the head, hunger signals can be at least partially blocked from being transmitted to the brain, thereby reducing hunger that can result in weight loss. In the electrode configuration where the anode is more distal to the head than the cathode is to the head, satiety signals can be transmitted from the electrodes to the brain along the vagus nerve, thereby also reducing hunger that can result in weight loss (although at a lesser rate than with the electrode configuration where the anode is more proximal to the head than the cathode is to the head).

Accordingly, in some embodiments the medical device 2101 is configured to at least partially control a rate of weight loss by being configured to control positions of the anode and the cathode relative to the head. If the rate of weight loss is too low while using the electrode configuration where the anode is more distal to the head than the cathode is to the head, then the polarities of the alpha electrode and the beta electrode can be switched so that the anode is more proximal to the head than the cathode is to the head, which can increase the weight loss rate. If the rate of weight loss is too high while using the electrode configuration where the anode is more proximal to the head than the anode is to the head, then the polarities of the alpha electrode and the beta electrode may be switched so that the anode is more distal to the head than the cathode is to the head, which can decrease the rate of weight loss.

In some other embodiments, the medical device 2101 is not configured to controllably set and change the polarity of the alpha electrode and the polarity of the beta electrode. For example, the alpha electrode may be fixedly configured to be an anode and the beta electrode may be fixedly configured to be a cathode, and the alpha electrode may be positionable (e.g., during implantation of the medical device 2101 in a person) along a vagus nerve of the person farther away from, or closer to, a head of the person compared to the beta electrode.

In some embodiments, the medical device 2101 may be configured to selectively drive the one or more first electrodes 2104A. This may provide, during stimulation, control of whether an anode (among electrodes of the one or more first electrodes 2104A being driven) is most proximal or distal to the head of the person compared to a cathode (among electrodes of the one or more first electrodes 2104A being driven).

For example, the one or more first electrodes 2104A may include an alpha electrode, a beta electrode, a gamma electrode, and a delta electrode. The alpha electrode may be fixedly configured to be an anode, the beta electrode may be fixedly configured to be a cathode, the gamma electrode may be fixedly configured to be a cathode, and the delta electrode may be fixedly configured to be an anode. The alpha electrode may be positionable along the vagus nerve more proximal to the head than the beta electrode may be, and the gamma electrode may be positionable along the vagus nerve more proximal to the head than the delta electrode may be. The medical device 2101 may be configured to control and drive the alpha electrode and the beta electrode independently from the gamma electrode and the delta electrode. The medical device 2101 may therefore be configured to drive the alpha electrode and the beta electrode without driving the gamma electrode and the delta electrode so that, among the first electrodes 2104A being driven, an anode is more proximal to the head than a cathode. The medical device 2101 may also be configured to drive the gamma electrode and the delta electrode without driving the alpha electrode and the beta electrode so that, among the first electrodes 2104A being driven, an anode is more distal to the head than a cathode. A rate of weight loss may therefore be at least partially controlled by selectively driving the first electrodes 2104A.

In some embodiments, the first electrodes 2104A may include three first electrodes sequentially arranged along the first electrical lead 2103A or along a cuff electrode. One of the three first electrodes may be positioned between the other two of the three first electrodes and is configured to be opposite (e.g., fixedly set to be opposite or controllably set to be opposite) in polarity compared to the other two of the three first electrodes.

In some embodiments, the one or more second electrodes 2104B may have any configuration that the one or more first electrodes 2104A may have, may be configurable in any manner that the one or more first electrodes 2104A may be configurable, may be controllable in any manner that the one or more first electrodes 2104A may be controllable, and/or may be drivable in any manner that the one or more first electrodes 2104A may be drivable. The one or more second electrodes 2104B may be the same or different in configuration as the one or more first electrodes 2104A, may be configurable in a same or different manner as the manner in which the one or more first electrodes 2104A are configurable in, may be controllable in a same or different manner as the manner in which the one or more first electrodes 2104A are controllable, and/or may be drivable in a same or different manner as the manner in which the one or more first electrodes 2104A are drivable.

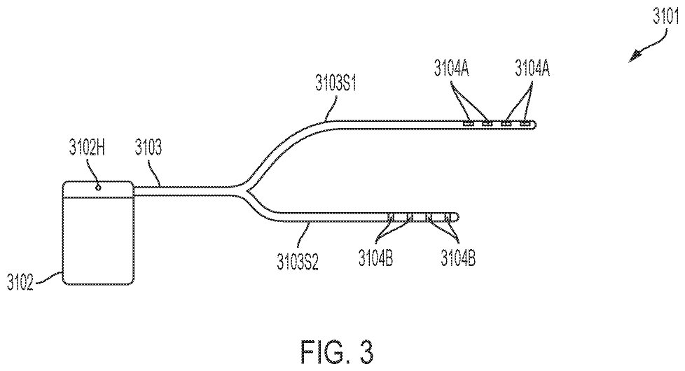

FIG. 3 illustrates a medical device 3101 according to some embodiments. The medical device 3101 may include some features similar to, or the same as, features of other medical devices within the scope of the present disclosure, for example, medical devices described hereinabove and medical devices described hereinbelow.

Referring to FIG. 3, the medical device 3101 may include a housing 3102 including a header 3102H, an electrical lead 3103 electrically coupled to the housing 3102, and a first sub electrical lead 3103S1 and a second sub electrical lead 3103S2 that are respectively electrically coupled to the housing 3102 via the electrical lead 3103. For example, the first and second sub electrical leads 3103S1 and 3103S2 may branch off from the electrical lead 3103.

The medical device 3101 may include one or more first stimulators coupled to the housing 3102 via the first sub electrical lead 3103S1 and configured to stimulate at least a first part of a vagus nerve of a person to treat at least one medical condition (e.g., obesity), and one or more second stimulators coupled to the housing 3102 via the second sub electrical lead 3103S2 and configured to stimulate a second part of the vagus nerve or another biological organ of the person to treat one or more medical conditions (e.g., one or more medical conditions comorbid with obesity). For example, the one or more first stimulators may include one or more first electrodes 3104A, and the one or more second stimulators may include one or more second electrodes 3104B.

By electrically coupling the first and second sub electrical leads 3101S1 and 3101S2 to the housing 3102 via the electrical lead 3103, invasion of the body (e.g., the amount of tunneling required by leads when implanting the medical device 3101) of a person may be reduced compared to if the medical device 3101 had two electrical leads separately coupled to the housing 3102 instead of the two sub electrical leads 3103S1 and 3103S2. The medical device 3101 including the first and second sub electrical leads 3101S1 and 3103S2 commonly coupled to the electrical lead 3103 may be desirable, for example, where a first location where the one or more first stimulators are configured to provide stimulation and a second location where the one or more second stimulators are configured to provide stimulation are sufficiently close together. In this case, much of the tunneling required to provide a pathway between housing 3102 and the first and second stimulators may be provided by a single pathway corresponding to the electrical lead 3103, instead of two separate pathways that could be required if the first and second stimulators were separately coupled to the housing 3102.

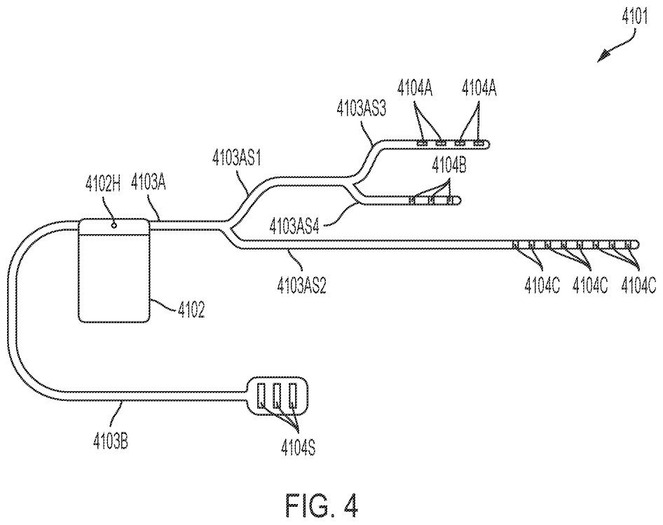

FIG. 4 illustrates a medical device 4101 according to some embodiments. The medical device 4101 may include some features similar to, or the same as, features of other medical devices within the scope of the present disclosure, for example, medical devices described hereinabove and medical devices described hereinbelow.

Referring to FIG. 4, the medical device 4101 may include a housing 4102 including a header 4102H, a first electrical lead 4103A electrically coupled to the housing 4102, and a second electrical lead 4103B electrically coupled to the housing 4102. The first and second electrical leads 4103A and 4103B may be separately coupled to the housing 4102. The medical device 4101 may include a first sub electrical lead 4103AS1 and a second sub electrical lead 4103AS2 that are respectively electrically coupled to the housing 4102 via the first electrical lead 4103A. For example, the first and second sub electrical leads 4103AS1 and 4103AS2 may branch off from the first electrical lead 4103A. The medical device 4101 may include a third sub electrical lead 4103AS3 and a fourth sub electrical lead 4103AS4 that are respectively electrically coupled to the housing 4102 through the first electrical sub lead 4103AS1. For example, the third and fourth sub electrical leads 4103AS3 and 4013AS4 may branch off from the first sub electrical lead 4104AS1.

The medical device 4101 may include one or more first stimulators (e.g., one or more first electrodes 4104A) coupled to the housing 4102 via the third sub electrical lead 4103AS3, one or more second stimulators (e.g., one or more second electrodes 4104B) coupled to the housing 4102 via the fourth sub electrical lead 4103AS4, one or more third stimulators (e.g., one or more third electrodes 4104C) coupled to the housing 4102 via the second sub electrical lead 4103AS2, and one or more sensors (e.g., one or more sensor electrodes 4104S) coupled to the housing 4102 via the second electrical lead 4103B. The one or more first stimulators, the one or more second stimulators, and the one or more third stimulators may be configured to treat one or more medical conditions, for example, obesity, both obesity and one or more medical conditions comorbid with the obesity, diabetes, both diabetes and one or more medical conditions comorbid with diabetes, OSA, or both OSA and one or more medical conditions comorbid with OSA. For example, one selected from among the one or more first stimulators, the one or more second stimulators, and the one or more third stimulators may be configured to provide stimulation to a first part of a person's vagus nerve to treat obesity, and the remaining two selected from among the one or more first stimulators, the one or more second stimulators, and the one or more third stimulators may be configured to provide stimulation to treat one or more other medical comorbid with the obesity.

The one or more sensors may be configured to sense (e.g., detect and/or measure) one or more biomarkers, one or more bioindicators, and/or one or more biophysical properties as disclosed herein. This information may be communicatively transmitted to a controller (e.g., microcontroller and/or microprocessor) or processor (e.g., in the housing 4102). The medical device 4101 may be configured (e.g., in response to the controller or processor of the medical device 4101 executing computer-readable instructions stored in a memory device in the medical device 4101) to alter the stimulation provided by the one or more first stimulators, the one or more second stimulators, and/or the one or more third stimulators based on the information received by the controller or processor from the one or more sensors.

For example, as explained herein, the medical device 4101 may be configured to provide first stimulation via the one or more first stimulators pursuant to a first treatment protocol, to provide second stimulation via the one or more second stimulators pursuant to a second treatment protocol, and to provide third stimulation via the one or more third stimulators pursuant to a third treatment protocol. The first, second, and third treatment protocols may be the same or different, and each of the first, second, and third treatment protocols may include electric current frequency, electric current amplitude, electric current pulse width, electric current duty cycle, electrode configuration (e.g., electrode polarity), a time duration for providing stimulation, and/or a time for an onset of providing stimulation. The medical device 4101 may be configured to modify at least one of the first treatment protocol, the second treatment protocol, or the third treatment protocol based on the information sensed by the one or more sensors.

As explained above, in some embodiments, a separate sensor or monitor may be included (e.g., in a medical system including the medical device 4101) that is not physically coupled to the medical device 4101. The separate sensor may be configured to sense any information disclosed herein that the one or more sensors coupled to the housing 4102 via the second electrical lead 4103B may be configured to sense. The separate sensor may be communicatively coupled (e.g., wirelessly communicatively coupled) to the medical device 4101 (e.g., to the controller or processor of the medical device 4101).

In some embodiments, a controller (e.g., a controller device) may be included (e.g., in a medical system including the medical device 4101) that is communicatively coupled to the medical device 4101 and is configured to receive information via one or more inputs. For example, the controller may include a user interface configured to receive input information (e.g., from a person receiving treatment from the medical device 4101 and/or from a physician), and the controller may be configured to transmit the information to the medical device 4101. In some embodiments, the controller may be configured to receive information relating to the person's weight (e.g., his or her current weight) and/or to the person's rate of weight change. The controller may be, for example, a phone, a tablet, or a watch. The medical device 4101 may be configured to modify the stimulation provided by the medical device 4101 (e.g., modify the first treatment protocol, the second treatment protocol, and/or the third treatment protocol) based on the information received from the controller.

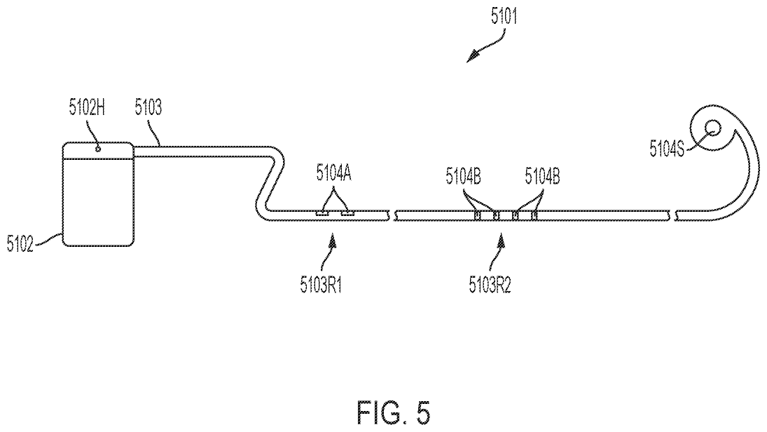

FIG. 5 illustrates a medical device 5101 according to some embodiments. The medical device 5101 may include some features similar to, or the same as, features of other medical devices within the scope of the present disclosure, for example, medical devices disclosed hereinabove and medical devices disclosed hereinbelow.

Referring to FIG. 5, the medical device 5101 may include a housing 5102 including a header 5102H, an electrical lead 5103 electrically coupled to the housing 5102, one or more first stimulators (e.g., one or more first electrodes 5104A) positioned on the electrical lead 5103 at a first region 5103R1, one or more second stimulators (e.g., one or more second electrodes 5104B) positioned on the electrical lead 5103 at a second region 5103R2, and one or more sensors (e.g., one or more sensor electrodes 5104S) positioned on the electrical lead 5103.

In some embodiments, the second region 5103R2 are spaced apart from the first region 5103R1 along the electrical lead 5103, and the one or more sensors are spaced apart from each of the first region 5103R1 and the second region 5103R2 along the electrical lead 5103. For example, the one or more sensors may be positioned at a distal end of the electrical lead 5103, the first region 5103R1 may be positioned between a proximal end of the electrical lead 5103 and the distal end of the electrical lead 5103, and the second region 5103R2 may be positioned between the first region 5103R1 and the distal end of the electrical lead 5103.

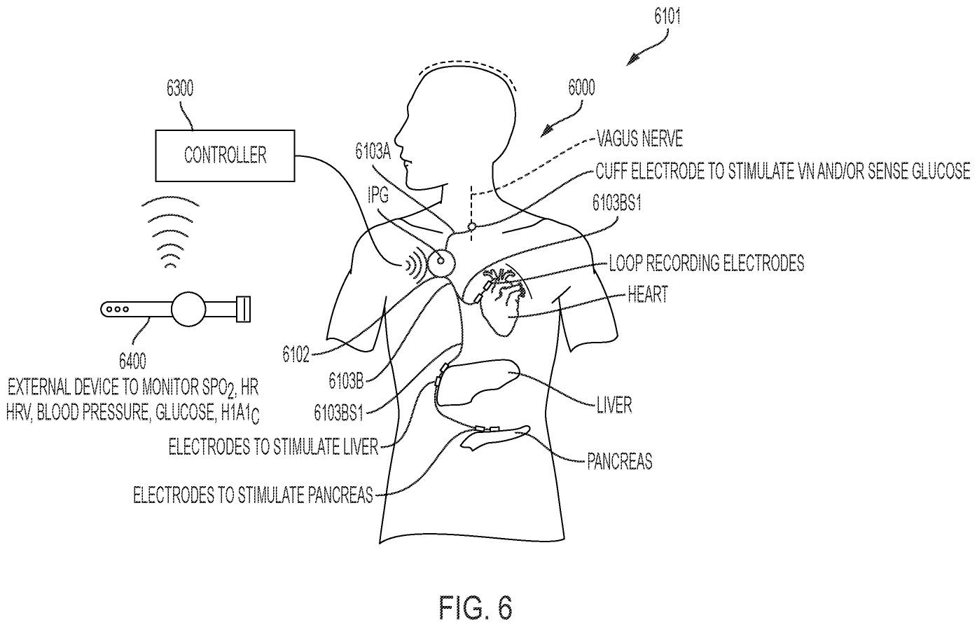

FIG. 6 illustrates a medical system 6000, including a medical device 6101 illustrated as being implanted in a person, according to some embodiments. The medical system 6000 may include some features similar to, or the same as, features of other medical systems within the scope of the present disclosure, for example, medical systems described hereinabove and medical systems described hereinbelow. The medical device 6101 may include some features similar to, or the same as, features of other medical devices within the scope of the present disclosure, for example, other medical devices described hereinabove and other medical devices described hereinbelow.

Referring to FIG. 6, the medical system 6000 may include the medical device 6101, a controller 6300 (e.g., a controller device) communicatively coupled (e.g., wirelessly communicatively coupled) to the medical device 6101, and a monitor 6400 (e.g., a sensor) configured to monitor one or more biomarkers, one or more bioindicators, and/or one or more biophysical properties.

The medical device 6101 may include a housing 6102, a first electrical lead 6103A electrically coupled to the housing 6102, a second electrical lead 6103B electrically coupled to the housing 6102, a first sub electrical lead 6103BS1 electrically coupled to the housing 6102 through the second electrical lead 6103B, and a second sub electrical lead 6103BS2 electrically coupled to the housing 6102 through the second electrical lead 6103B. For example, the first and second sub electrical leads 6103BS1 and 6103BS2 may branch from the second electrical lead 6103B.

The medical device 6101 may include one or more first stimulators (e.g., one or more first electrodes, such as a cuff electrode) electrically coupled to the first electrical lead 6103A, one or more second stimulators (e.g., one or more second electrodes, such as one or more loop recording electrodes) electrically coupled to the first electrical sub lead 6103BS1, one or more third stimulators (e.g., one or more third electrodes) electrically coupled to the second sub electrical lead 6103BS2 at a first region of the second sub electrical lead 6103BS2, and one or more fourth stimulators (e.g., one or more electrodes) electrically coupled to the second sub electrical lead 6103BS2 at a second region of the second sub electrical lead 6103BS2.

The medical device 6101 may be configured to treat one or more medical conditions by providing first stimulation to first biological tissue via the one or more first stimulators and pursuant to a first treatment protocol, providing second stimulation to second biological tissue via the one or more second stimulators and pursuant to a second treatment protocol, providing third stimulation to third biological tissue via the one or more third stimulators and pursuant to a third treatment protocol, and/or providing fourth stimulation to fourth biological tissue via the one or more fourth stimulators and pursuant to a fourth treatment protocol. In the non-limiting depicted embodiment, the first biological tissue is the vagus nerve, the second biological tissue is the heart, the third biological tissue is the liver, and the fourth biological tissue is the pancreas.

The medical device 6101 may include one or more sensors located on at least one of the first electrical lead 6103A, the first sub electrical lead 6103BS1, the second sub electrical lead 6103BS2, or another electrical lead or sub electrical lead coupled to the housing 6102. The one or more sensors may be configured to sense one or more biomarkers, one or more bioindicators, and/or one or more biophysical properties. In some embodiments, the one or more sensors may be on (e.g., directly on) the housing 6102 or in the housing 6102. In some embodiments, the medical system 6000 may include one or more sensors on a separate device. The separate device may be an implantable device separate from the medical device 6101 and communicatively coupled (e.g., wirelessly communicatively coupled) to the medical device 6101 or a device operable at least partially outside of the person and communicatively coupled (e.g., wirelessly communicatively coupled) to the medical device 6101. The separate device may be configured to transmit sensed (e.g., detected and/or measured) information relating to the one or more biomarkers, one or more bioindicators, and/or the one or more biophysical properties, and the medical device 6101 may be configured to control stimulation (e.g., modify the stimulation, for example, by modification one or more treatment protocols) provided by the medical device 6101 based on the sensed information.

The one or more medical conditions that the medical device 6101 may be configured to treat may include, for example, obesity, both obesity and one or more medical conditions comorbid with obesity, diabetes, both diabetes and one or more medical conditions comorbid with diabetes, OSA, or both OSA and one or more medical conditions comorbid with OSA.

Diabetes is a disease that results in poor glycemic regulation of the bloodstream and too much glucose in the body. Diabetes can be caused by the immune system attacking cells that produce insulin (type I diabetes), poor insulin production and/or poor insulin sensitivity (type II diabetes), and/or too much glucose resulting from a person's diet and/or overproduction of glucose by the person's liver. In some embodiments, diabetes can be treated, in part, by insulin therapy (e.g., monitoring a person's blood glucose and/or HbA1c, and injecting insulin into the person). In addition or alternatively, in some embodiments, diabetes can be treated by stimulating one or more targets (e.g., nerves, muscles, and/or organs) of the body that contribute to diabetes and poor glycemic control.

For example, providing stimulation to the stomach (e.g., gastric pacing and/or vagal pacing of the stomach) can slow gastric emptying, and thus, slow the release of food borne sugar and glucose by-products into the blood, while also increasing the sense of satiety and thereby reducing overall food consumption and glucose release. Providing stimulation to activate or block one or more vagal nerves innervating organs such as the abdomen can decrease weight and increase glycemic control. Stimulating the carotid sinus (e.g., with blocking high frequency of AC signal) can increase insulin sensitivity and glucose tolerance. Stimulating the pancreatic sympathetic nerve that projects to the lymph nodes can reduce progression of Type I diabetes. Stimulating Parenchyma tissue of the liver can reduce glucose production, slow gastric emptying, and increase GLP-1. Stimulating the pancreas can modulate insulin and glucagon production. Stimulating the vagus nerve can reduce the production of TNF that contributes to inflammation and insulin resistance. Stimulating the spine and/or the vagus nerve may reduce the sensation of pain caused by diabetic neuropathy. In some embodiments, targets for stimulation may include afferents or efferents of the vagus nerve that innervate organs and/or muscles, such as the stomach, liver, abdomen, and/or pancreas; the carotid sinus nerve; the pancreatic sympathetic nerve; the pancreas tissue itself; the liver (e.g., the parenchyma tissue of the liver); nerves innervating the liver (e.g., hepatic nerves); and the upper intestine.

Thus, diabetes can be treated by providing stimulation to any one of these targets. However, because stimulation of different ones of these targets may treat different aspects of diabetes (e.g., glucose production, insulin sensitivity, neuropathy pain, gastric emptying, etc.), and because stimulation of one of these targets may only partially treat the one or more aspects of diabetes that such stimulation treats, stimulating only one of these targets may not provide desirable treatment results. Accordingly, in some embodiments, a medical system (or medical device) configured to provide stimulation to multiple ones of these targets is provided, and thus, can provide a more comprehensive and/or thorough treatment of diabetes compared to medical systems or medical devices that are configured to only stimulate one target for treating diabetes. In some embodiments, the medical system 6000 is configured to provide stimulation to modulate pain related to diabetes, modulate endogenous insulin production, modulate insulin sensitivity, and/or modulate glycemic control.

Although the medical device 6101 is depicted as an implantable device, in some other embodiments, the present disclosure is not limited thereto. The medical system may include one or more implantable medical devices configured to provide stimulation to one or more targets for treating diabetes from within a person to be treated, and/or one or more medical devices configured to provide stimulation to one or more of targets for treating diabetes from without the person to be treated. For example, acoustic, electric, and/or inductive stimulation may be provided to one or more targets for treating diabetes from the one or more medical devices outside of the person and through the skin of the person.

In some embodiments, the medical device 6101 is configured to provide insulin to the person and/or to provide to the person other medications to control diabetes, glycemia, and/or pain. For example, the medical device may include an insulin or medication pump. In some other embodiments, the medical system 6000 includes a second medical device (e.g., a medication pump) separate from the medical device 6101 and configured to provide the insulin or other medication. In some methods of treating diabetes, the method may include providing stimulation to one or more targets of a person as described herein, and providing insulin and/or other medication to at least partially control the diabetes, glycemia, and/or pain.

In some embodiments, a controller (e.g., microcontroller and/or microprocessor) or a processor of the medical device 6101 (or of another device, such as the controller 6300) may be configured to identify which targets to provide stimulation to from among the targets that the medical device 6101 is configured to provide stimulation to. The controller or processor may be configured to identify such targets at least in part based on information sensed by the one or more sensors of the medical system 6000 or the medical device 6101.

In some embodiments, the medical system 6000 (e.g., the medical device 6101) may be configured for machine learning because the medical system 6000 may be configured to provide stimulation to several targets, may include several sensor inputs, and/or may be configured to provide a variety of stimuli (e.g., a variety of types of stimulation).

Although some systems and devices configured to treat, and methods for treating, diabetes by providing stimulation to multiple targets to achieve a synergistic affect compared to stimulating a single target have been illustrated and described with respect to FIG. 6, this is merely an example, and the present disclosure includes other systems, devices, and methods for treating, other medical conditions by providing stimulation to multiple targets to provide a more comprehensive and thorough treatment for such medical conditions. For example, the present disclosure includes systems and devices configured to treat, and methods for treating, obesity, cardiac issues, oncology, movement disorders, pain and spasticity, substance abuse and/or withdrawal, addiction, inflammatory disease, sleep apnea, OSA, CSA, and other medical conditions.

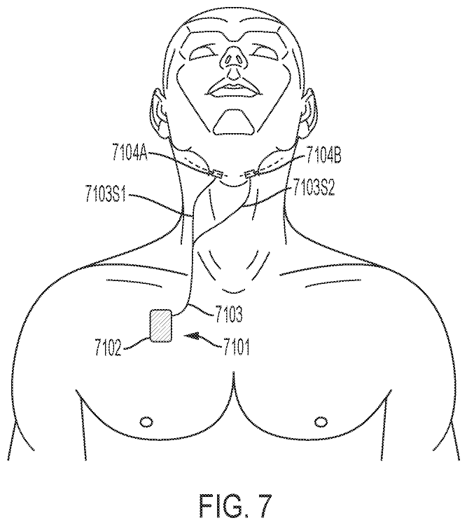

FIG. 7 illustrates a medical device 7101 according to some embodiments and implanted in a person. The medical device 7101 may include some features similar to, or the same as, features of other medical devices withing the scope of the present disclosure, for example, medical devices described hereinabove and medical devices described hereinbelow.

Referring to FIG. 7, the medical device 7101 may include a housing 7102, an electrical lead 7103 electrically coupled to the housing 7102, a first sub electrical lead 7103S1 electrically coupled to the housing 7102 through the electrical lead 7103, and a second sub electrical lead 7103S2 electrically coupled to the housing 7102 through the electrical lead 7103. For example, the first and second sub electrical leads 7103S1 and 7103S2 may branch from the electrical lead 7103.

The medical device 7101 may have one or more first stimulators (e.g., one or more first electrodes, such as one, two, three, four, five, or six or more electrodes; one or more magnetic stimulators; and/or one or more vibratory stimulators) configured to provide first stimulation to a right branch or a left branch of a person's hypoglossal nerve (HGN), and one or more second stimulators (e.g., one or more second electrodes, such as one, two, three, four, five, or six or more electrodes; one or more magnetic stimulators; and/or one or more vibratory stimulators) configured to provide second stimulation to the other branch of the person's HGN or to another organ (e.g., another nerve) such as the vagus nerve or the phrenic nerve. In some embodiments, the one or more first stimulators are coupled to the first sub electrical lead 7103S1 and may include a first cuff electrode 7104A. The one or more second stimulators may be coupled to the second sub electrical lead 7103S2 and may include a second cuff electrode 7104B.

The medical device 7101 may be configured to provide the first stimulation in accordance with first treatment protocol and to provide the second stimulation in accordance with a second treatment protocol that may be the same as, or different from the first treatment protocol. In some embodiments, the medical device 7101 may include two electrical leads separately electrically coupled to the housing 7102 instead of two sub electrical leads commonly electrically coupled to the housing 7102 through a common electrical lead.

The medical device 7101 may be configured to treat one or more medical conditions via providing the first stimulation and the second stimulation. For example, the medical device 7101 may be configured to treat OSA or both OSA and one or more medical conditions comorbid with the OSA.

Because the medical device 7101 may have multiple sets of one or more stimulators configured to respectively provide stimulation to multiple targets of the person to be treated for OSA, the medical device 7101 may be configured to yield superior treatment results compared to a medical device configured to provide stimulation to only one target of the person for treating OSA. For example, the medical device 7101 may be configured to provide stimulation to both the left branch and the right branch of the person's HGN, which can provide better treatment of OSA in persons having a high body mass index (BMI) compared to if only the left or right branch of the person's HGN was stimulated. Additionally, a medical device configured to provide bi-lateral stimulation (e.g., to both the left and right branches of the HGN) may be configured to alternate in time stimulation between left and right stimulation, which can reduce muscle fatigue in the person being treated. Furthermore, a medical device configured to provide stimulation to multiple targets may be configured to treat not only OSA, but also one or more medical conditions comorbid with the OSA and that may be caused by, or aggravated by, the OSA, thereby enabling treatment of multiple related conditions. Medical conditions comorbid with OSA can include central sleep apnea (CSA), hypertension, obesity, depression, gastroesophageal reflux disease (GERD), diabetes mellitus, hypercholesterolemia, asthma. At least some of these comorbidities may be treatable via stimulation of at least one of a vagus nerve, a baroreceptor, a splenic nerve, or a carotid sinus.

In the non-limiting depicted embodiment, the one or more first stimulators include a first cuff electrode 7104A at a distal end of the first sub electrical lead 7103S1 and configured to provide stimulation to the right branch of the person's HGN (e.g., a distal portion of the right branch of the HGN), and the one or more second stimulators include a second cuff electrode 7104B at a distal end of the second sub electrical lead 7103S2 and configured to provide stimulation to the left branch of the person's HGN (e.g., a distal portion of the left branch of the HGN). Each of the first and second cuff electrodes 7104A and 7104B may include one or more electrodes. For example, the first and second cuff electrodes 7104A and 7104B may each be a three-electrode cuff electrode configured to provide stimulation through each of three electrodes in the cuff electrode. In some embodiments, each of the three electrodes of the cuff electrode may be independently drivable and controllably set to be either an anode or a cathode, as explained in more detail above. In some embodiments, the three electrodes are arrangeable along a nerve or organ so that a middle electrode of the three electrodes is between the other two electrodes of the three electrodes, and the middle electrode can be configured to have a polarity opposite to the polarity of the other two electrodes of the three electrodes. The medical device 7101 may therefore be configured to direct an anodal block either up or down the nerve or organ (e.g., the vagus nerve or the HGN), or tripolar stimulation could be provided. In some embodiments, the medical device is configured to provide stimulation through the cuff electrode with sufficiently low duty cycle that afferent stimulation and efferent stimulation can be provided by the same cuff electrode. Each of the first and second cuff electrodes 7104S1 and 7104S2 may be configured (e.g., shaped and/or sized) for the distal portions of the right and left branches of the HGN, respectively.

In some embodiments, the first and second sub electrical leads 7103S1 and 7103S2 may be different, or substantially the same, in size (e.g., length). For example, the first and second sub electrical leads 7103S1 and 7103S2 may have different lengths, which can allow for the one or more first stimulators to be positionable to target a different portion of the HGN than the one or more second stimulators, allow for the one or more first stimulators to be positionable to target a different nerve than the one or more second stimulators, and/or allow the electrical lead 7103 to be positionable to a side of a center of the person's neck before branching off into the first and second sub electrical leads 7103S1 and 7103S2.

The medical device 7101 may be implantable within a person such that the housing 7102 is positionable in the person's chest. The electrical lead 7103 may be configured (e.g., shaped and/or sized) to lead from the housing 7102 to the person's neck before bifurcating into the first and second sub electrical leads 7103S1 and 7103S2. In some embodiments, the position of each of the first and second sub electrical leads 7103S1 and 7103S2 may be substantially fixable via a respective suture anchor.

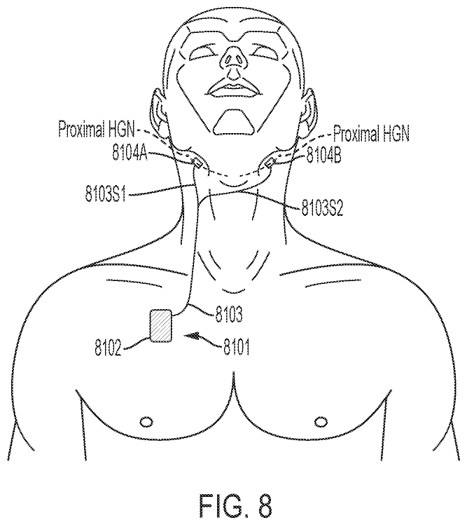

FIG. 8 illustrates a medical device 8101 according to some embodiments and implanted in a person. The medical device 8101 may include some features similar to, or the same as, features of other medical devices within the scope of the present disclosure, for example, medical devices described hereinabove and medical devices described hereinbelow.

Referring to FIG. 8, the medical device 8101 may include a housing 8102, an electrical lead 8103 electrically coupled to the housing 8102, a first sub electrical lead 8103S1 electrically coupled to the housing 8102 through the electrical lead 8103, and a second sub electrical lead 8103S2 electrically coupled to the housing 8102 through the electrical lead 8103. For example, the first and second sub electrical leads 8103S1 and 8103S2 may branch from the electrical lead 8103.

The medical device 8101 may include one or more first stimulators coupled to the first sub electrical lead 8103S1 and configured to provide first stimulation to a first target, and one or more second stimulators coupled to the second sub electrical lead 8103S2 and configured to provide second stimulation to a second target. In the non-limiting depicted embodiment, the one or more first stimulators include a first cuff electrode 8104A configured to provide the first stimulation to a proximal portion of the right branch of the person's HGN, and the one or more second stimulators include a second cuff electrode 8104B configured to provide the second stimulation to a proximal portion of a left branch of the person's HGN. For example, the first and second cuff electrodes 8104A and 8104B may each be shaped, sized, and/or have a high enough number of electrodes (e.g., six or more electrodes) to respectively provide suitable stimulation to the proximal portions of the right and left branches of the HGN.

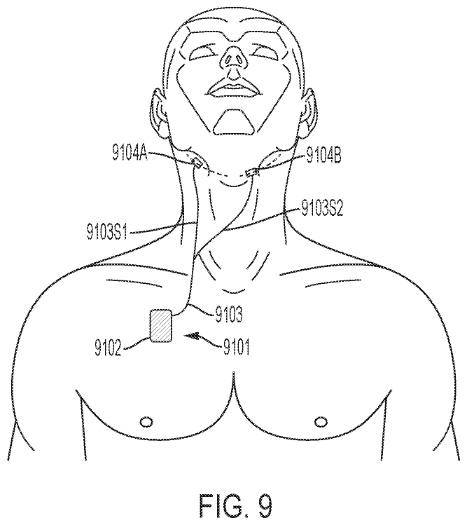

FIG. 9 illustrates a medical device 9101 according to some embodiments and implanted in a person. The medical device 9101 may include some features similar to, or the same as, features of other medical devices within the scope of the present disclosure, for example, medical devices described hereinabove and medical devices described hereinbelow.

Referring to FIG. 9, the medical device 9101 may include a housing 9102, an electrical lead 9103 electrically coupled to the housing 9102, a first sub electrical lead 9103S1 electrically coupled to the housing 9102 through the electrical lead 9103, and a second sub electrical lead 9103S2 electrically coupled to the housing 9102 through the electrical lead 9103. For example, the first and second sub electrical leads 9103S1 and 9103S2 may branch from the electrical lead 9103.

The medical device 9101 may include one or more first stimulators coupled to the first sub electrical lead 9103S1 and configured to provide first stimulation to a first target, and one or more second stimulators coupled to the second sub electrical lead 9103S2 and configured to provide second stimulation to a second target. In the non-limiting depicted embodiment, the one or more first stimulators include a first cuff electrode 9104A configured to provide the first stimulation to a proximal portion of the right branch of the person's HGN, and the one or more second stimulators include a second cuff electrode 9104B configured to provide the second stimulation to a distal portion of a left branch of the person's HGN. In some other embodiments, the one or more first stimulators may include a first cuff electrode configured to provide the first stimulation to the proximal portion of the right branch (or left branch) of the HGN and the one or more second stimulators may include a second cuff electrode configured to provide the second stimulation to a distal portion of the right branch (or left branch) of the HGN.

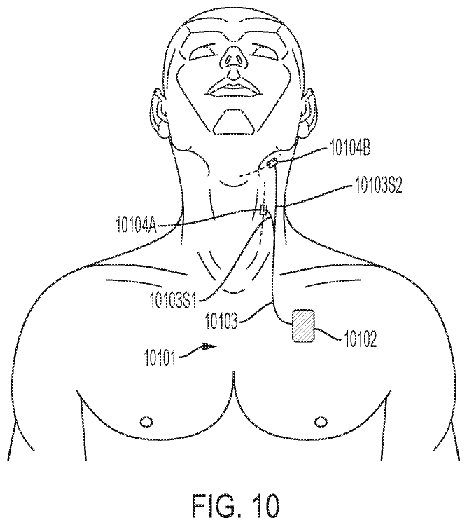

FIG. 10 illustrates a medical device 10101 according to some embodiments and implanted in a person. The medical device 10101 may include some features similar to, or the same as, features of other medical devices within the scope of the present disclosure, for example, medical devices described hereinabove and medical devices described hereinbelow.

Referring to FIG. 10, the medical device 10101 may include a housing 10102, an electrical lead 10103 electrically coupled to the housing 10102, a first sub electrical lead 10103S1 electrically coupled to the housing 10102 through the electrical lead 10103, and a second sub electrical lead 10103S2 electrically coupled to the housing 10102 through the electrical lead 10103. For example, the first and second sub electrical leads 10103S1 and 10103S2 may branch from the electrical lead 10103.

The medical device 10101 may include one or more first stimulators coupled to the first sub electrical lead 10103S1 and configured to provide first stimulation to a first target, and one or more second stimulators coupled to the second sub electrical lead 10103S2 and configured to provide second stimulation to a second target. In the non-limiting depicted embodiment, the one or more first stimulators include a first cuff electrode 10104A configured to provide the first stimulation to the left branch of the person's HGN (e.g., a proximal portion or a distal portion of the left branch of the HGN), and the one or more second stimulators include a second cuff electrode 10104B configured to provide the second stimulation to a left side of the person's vagus nerve. In some other embodiments of the medical device 10101, the one or more second stimulators may be configured to provide stimulation to at least one of the vagus nerve, a carotid sinus, a baroreceptor, a phrenic nerve (e.g., a terminal portion of the phrenic nerve), a splenic nerve, or a body of a diaphragm muscle. The medical device 10101 may therefore be configured to treat at least one of OSA, CSA, heart failure, obesity, depression, epilepsy, or rheumatoid arthritis.

In some embodiments, the medical device 10101 may include one or more sensors (e.g., one or sensor electrodes) electrically coupled to the housing 10102 through an electrical lead (e.g., a sub electrical lead or a main electrical lead) and configured to be positioned in proximity with the vagus nerve and to sense information relating to (e.g., occurrence and/or timing of) respiration of the person. In some other embodiments, the one or more sensors may be configured to be positioned in proximity with the phrenic nerve or with a muscle enervated by the phrenic nerve, and to sense information relating to respiration (e.g., to sense local field potential). The medical device 10101 may be configured to utilize such information relating to respiration to control stimulation provided to the HGN (e.g., to control timing of the stimulation provided to the HGN). For example, the medical device 10101 may be configured to synchronize stimulation applied to the HGN with the person's inspiration.

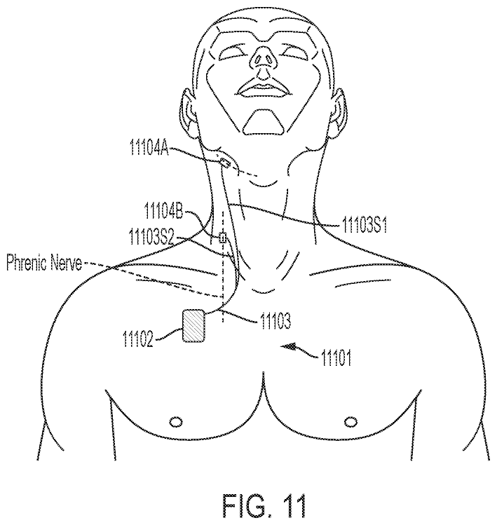

FIG. 11 illustrates a medical device 11101 according to some embodiments and implanted in a person. The medical device 11101 may include some features similar to, or the same as, features of other medical devices within the scope of the present disclosure, for example, medical devices described hereinabove and medical devices described hereinbelow.

Referring to FIG. 11, the medical device 11101 may include a housing 11102, an electrical lead 11103 electrically coupled to the housing 11102, a first sub electrical lead 11103S1 electrically coupled to the housing 11102 through the electrical lead 11103, and a second sub electrical lead 11103S2 electrically coupled to the housing 11102 through the electrical lead 11103. For example, the first and second sub electrical leads 11103S1 and 11103S2 may branch from the electrical lead 11103.

The medical device 11101 may include one or more first stimulators coupled to the first sub electrical lead 11103S1 and configured to provide first stimulation to a first target, and one or more second stimulators coupled to the second sub electrical lead 11103S2 and configured to provide second stimulation to a second target. In the non-limiting depicted embodiment, the one or more first stimulators include a first cuff electrode 11104A configured to provide the first stimulation to the right branch of the person's HGN (e.g., a proximal portion or a distal portion of the right branch of the HGN), and the one or more second stimulators include a second cuff electrode 11104B configured to provide the second stimulation to the person's phrenic nerve.

The vagus nerve and the phrenic nerve may each be stimulated to modulate the “fight or flight” response of the sympathetic nervous system. Thus, it is possible to achieve with stimulation of the phrenic nerve at least some of the same beneficial effects that can be achieved with stimulation of the vagus nerve. In some embodiments, the one or more second stimulators are configured to stimulate (e.g., to controllably stimulate) afferent fibers of the phrenic nerve. The one or more second stimulators may be configured to selectively stimulate afferent fibers of the phrenic nerve (e.g., fibers of the phrenic nerve for sensing). For example, the one or more second stimulators may be configured to stimulate afferent fibers of the phrenic nerve without substantially stimulating efferent fibers of the phrenic nerve (e.g., fibers of the phrenic nerve for motor functions). In some embodiments, the medical device 11101 is configured to selectively (e.g., independently) drive the one or more second stimulators, and the one or more second stimulators include a plurality of electrodes that have a number high enough to allow the medical device 11101 to selectively stimulate afferent fibers of the phrenic nerve. Selective stimulation of the afferent fibers of the phrenic nerve can allow for modulation of the sympathetic nervous system without substantially affecting breathing.

In some embodiments, the medical device 11101 may be configured (e.g., via one or more treatment protocols) to by provide stimulation to the phrenic nerve with frequencies and/or waveforms that block nerve conduction of afferent fibers, rather than inducing nerve conduction of the afferent fibers. Such stimulation can provide modulation of the sympathetic nervous system without substantially affective breathing of the person.

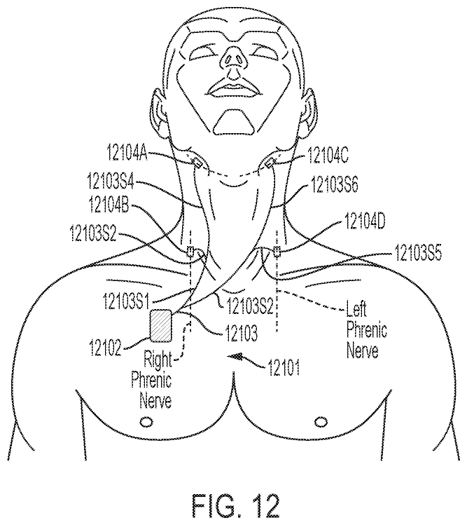

FIG. 12 illustrates a medical device 12101 according to some embodiments and implanted in a person. The medical device 12101 may include some features similar to, or the same as, features of other medical devices within the scope of the present disclosure, for example, medical devices described hereinabove and medical devices described hereinbelow.

Referring to FIG. 12, the medical device 12101 may include a housing 12102, an electrical lead 12103 electrically coupled to the housing 12102, a first sub electrical lead 12103S1 electrically coupled to the housing 11102 through the electrical lead 11103, a second sub electrical lead 12103S2 electrically coupled to the housing 11102 through the electrical lead 12103, third and fourth sub electrical leads 1203S3 and 12103S4 respectively electrically coupled to the housing 12102 through the first sub electrical lead 12103S1, and fifth and sixth sub electrical leads 1203S5 and 1203S6 respectively electrically coupled to the housing 12102 through the second sub electrical lead 12103S2.

The medical device 12101 may include one or more first stimulators coupled to the fourth sub electrical lead 12103S4 and configured to provide first stimulation to a first target (e.g., a right branch of an HGN of the person), one or more second stimulators coupled to the third sub electrical lead 12103S3 and configured to provide second stimulation to a second target (e.g., a right phrenic nerve of the person), one or more third stimulators coupled to the sixth sub electrical lead 12103S6 and configured to provide third stimulation to a third target (e.g., a left branch of the HGN), and one or more fourth stimulators coupled to the fifth sub electrical lead 12103S5 and configured to provide fourth stimulation to a fourth target (e.g., a left phrenic nerve of the person). In the non-limiting depicted embodiment, the one or more first stimulators include a first cuff electrode 12104A, the one or more second stimulators include a second cuff electrode 12104B, the one or more third stimulators include a third cuff electrode 12104C, and the one or more fourth stimulators include a fourth cuff electrode 12104D.

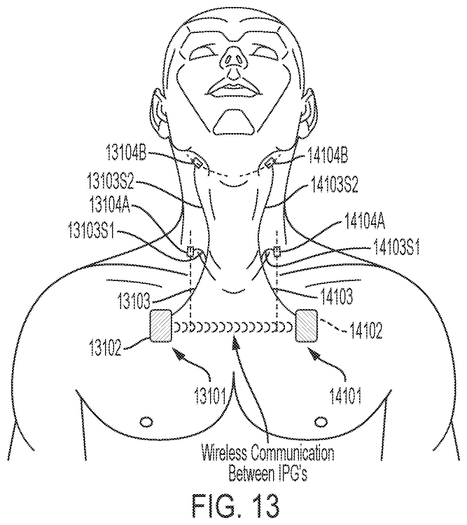

FIG. 13 illustrates a medical system according to some embodiments. The medical system may include a first medical device 13101 and a second medical device 14101. The medical system may include some features similar to, or the same as, features of other medical systems within the scope of the present disclosure, for example, medical systems described hereinabove and medical systems described hereinbelow. Each of the first and second medical devices 13101 and 14101 may include some features similar to, or the same as, features of other medical devices within the scope of the present disclosure, for example, medical devices described hereinabove and medical devices described hereinbelow.

Referring to FIG. 13, the first medical device 13101 may include a housing 13102, an electrical lead 13103 electrically coupled to the housing 13102, first and second sub electrical leads 13103S1 and 13103S2 electrically coupled to the housing 13102 through the electrical lead 13102, one or more first stimulators (e.g., one or more first electrodes 13104A) coupled to the first sub electrical lead 13103S1, and one or more second stimulators (e.g., one or more second electrodes 13104B) coupled to the second sub electrical lead 13103S2. The second medical device 14101 may include a housing 14102, an electrical lead 14103 electrically coupled to the housing 14102, first and second sub electrical leads 14103S1 and 14103S2 electrically coupled to the housing 14102 through the electrical lead 14102, one or more first stimulators (e.g., one or more first electrodes 14104A) coupled to the first sub electrical lead 14103S1, and one or more second stimulators (e.g., one or more second electrodes 14104B) coupled to the second sub electrical lead 14103S2.

In some embodiments, the first and second medical devices 13101 and 14101 may be communicatively coupled (e.g., wirelessly communicatively coupled) to each other and/or to an external controller device, for example, by Bluetooth and/or near field communication (NFC). For example, the first and second medical devices 13101 and 14101 may be configured to communicate with each other either directly or through one or more other devices, such as the external controller device. The medical system may be configured to control stimulation provided by the first and second medical devices 13101 and 14101 based on information transmitted by the first and second medical devices 13101 and 14101. For example, the stimulation provided by the first and second medical devices 13101 and 14101 may be coordinated (e.g., synchronized) based on the information provided by the first and second medical devices 13101 and 14101.

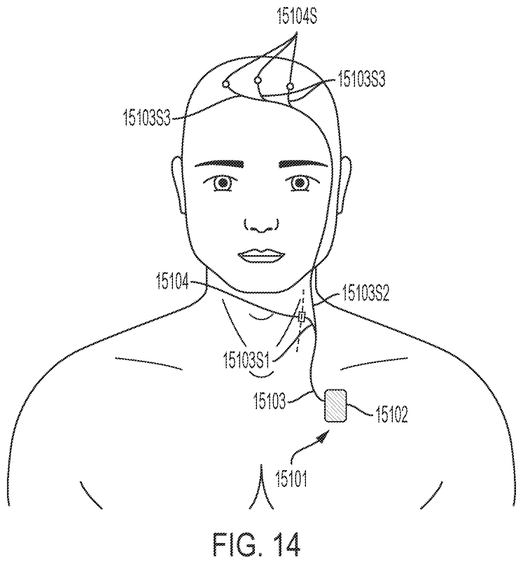

FIG. 14 illustrates a medical device 15101 according to some embodiments. The medical device 15101 may include some features similar to, or the same as, features of other medical devices within the scope of the present disclosure, for example, medical devices described hereinabove and medical devices described hereinbelow.

Referring to FIG. 14, the medical device may 15101 may include a housing 15102, an electrical lead 15103 electrically coupled to the housing 15102, first and second sub electrical leads 15103S1 and 15103S2 electrically coupled to the housing 15102 through the electrical lead 15103, a plurality of third sub electrical leads 15103S3 electrically coupled to the housing 15102 through the second sub electrical lead 15103S2, one or more first stimulators (e.g., one or more first electrodes 15104) coupled to the first sub electrical lead 15103S1, and a plurality of sensor electrodes 1504S (e.g., a plurality of subdermal sensor electrodes) coupled to the third sub electrical lead 15103S3. For example, the plurality of sensor electrodes 1504S may be configured to record electroencephalogram (EEG) and/or may be configured to detect when an epileptic event is occurring or is about to occur.

Information sensed by the plurality of sensor electrodes may be transmitted to a controller (e.g., microcontroller and/or microprocessor) or processor (e.g., in the medical device 15101), and the medical device 15101 may be configured to control (e.g., modify) stimulation provided by the one or more first stimulators 15104 based on the information sensed by the plurality of sensor electrodes 1504S. For example, the plurality of sensor electrodes 1504S may be configured to sense information relating to a seizure, and the medical device 15101 may be configured to boost stimulation through the one or more first stimulators (e.g., to the vagus nerve) in response to, and based on, the information relating to the seizure to mitigate the seizure. In some embodiments, for sensitive seizure detection and/or electrocorticography recording, small openings could be made in the skull to allow placement of the plurality of sensors on the cortex.

Although the discussion of medical systems and medical devices depicted in FIGS. 7-14 has related in part to treating OSA and to stimulating a right or left branch of the HGN, the present disclosure is not limited thereto. Medical systems and medical devices within the scope of the present disclosure, including medical devices and medical systems described with reference to FIGS. 7-14, may be configured in some embodiments to treat heart failure or both heart failure and one or more of epilepsy, major depressive disorder, and rheumatoid arthritis. For example, medical systems or medical devices may be configured to provide one or more of bilateral stimulation to the vagus nerve, stimulation of the splenic nerves, and stimulation of the phrenic nerves.

In some embodiments, a medical system or medical device is configured to treat diabetes and to use a bifurcated or dual lead to provide bilateral stimulation to the vagus nerve. In some embodiments, one set of one or more stimulators (e.g., on one lead) may be positionable on a vagal branch going to a pancreas, and another set of one or more stimulators (e.g., on another lead) may be positionable on a vagal branch going to a liver.

In some embodiments, a medical system or medical device is configured to treat epilepsy and to use a bifurcated or dual lead to provide stimulation to a left portion of the vagus nerve and to a right portion of the vagus nerve. The medical system and/or medical device may be configured to provide stimulation to the left portion of the vagus nerve that is either continuous or periodic based on a duty cycle and that provides a boost in amplitude in response to detection (e.g., by a sensor of the medical system) of ictal tachycardia to prevent a seizure from occurring. The medical system and/or medical device may be configured to provide stimulation to the right portion of the vagus nerve to resolve the ictal tachycardia or bradycardia.

In some embodiments, any electrode of a medical system and/or medical device within the scope of the present disclosure may be configured to either stimulate or record nerve activity (e.g., activity of the HGN, VNS, etc.), and the medical system and/or medical device may be configured to reconfigure (e.g., by a controller (e.g., a microcontroller and/or a microprocessor) or processor in response to executing computer readable instructions stored in a memory device) any electrode between being configured to provide stimulation and being configured to sense information. For example, in a medical system and/or medical device including first and second cuff electrodes, the first cuff electrode could be configured to provide stimulation, and the second cuff electrode could be configured to record or map nerve activity during initial surgery. In some embodiments, after the initial surgery is completed, the second cuff electrode could be reconfigured (e.g., reprogrammed by a controller (e.g., a microcontroller and/or a microprocessor) or processor in response to executing computer readable instructions stored in a memory device) to provide stimulation. In some embodiments, the first and/or second cuff electrodes may be reprogrammed from being configured to provide stimulation to being configured to record or map nerve activity.

Methods for treating one or more medical conditions (e.g., obesity, diabetes, OSA, and comorbidities thereof) are included within the scope of the present disclosure. In some embodiments, a method may be performed utilizing any medical system and/or medical device within the scope of the present disclosure. In some embodiments, a method may include any combination of tasks that the medical systems and/or medical devices within the scope of the present disclosure are configured to perform, and the tasks in the combination of tasks may be temporally ordered in any order.

As a non-limiting example, some medical devices are described herein as being configured to provide stimulation via an alpha electrode and a beta electrode and to controllably reverse the polarities of the alpha electrode and of the beta electrode. Therefore, some methods for treating one or more medical conditions that are within the scope of the present disclosure include a task of setting the alpha electrode as a cathode and a beta electrode as an anode, a task providing electrical stimulation while the alpha electrode and the beta electrode are respectively set to be the cathode and the anode, a task of reversing the polarities of the alpha electrode and the beta electrode so that the alpha electrode is an anode and the beta electrode is a cathode, and a task of providing electrical stimulation while the alpha electrode and the beta electrode are respectively set to be the anode and the cathode.

While the present invention has been described in connection with certain example embodiments, it is to be understood that the invention is not limited to the disclosed embodiments, but, on the contrary, is intended to cover various modifications and equivalent arrangements included within the spirit and scope of the appended appendix, claims, and equivalents thereof.

What is claimed is:

1. A medical device, comprising:
one or more stimulation leads configured to stimulate at least a person's vagus nerve to treat, via the stimulation of the person's vagus nerve, both obesity and one or more medical conditions comorbid with the obesity; and
a controller coupled to the one or more stimulation leads and configured to control and drive the stimulation provided by the one or more stimulation leads.

2. The medical device of claim 1, wherein the one or more stimulation leads comprise a first electrode and a second electrode, the first and second electrodes being configured to provide electric stimulation to the person's vagus nerve and to be positionable so that the first electrode is more proximal to a head of the person than the second electrode is to the person's head.

3. The medical device of claim 2, wherein the first and second electrodes are configured to be controllably changeable between at least a first configuration, in which the first electrode is a cathode and the second electrode is an anode, and a second configuration, in which the first electrode is an anode and the second electrode is a cathode.

4. The medical device of claim 2, further comprising a third and fourth electrode, the third and fourth electrodes being configured to provide electric stimulation to the person's vagus nerve and to be positionable so that the third electrode is more proximal to the person's head than the fourth electrode is,
wherein the first electrode and the fourth electrodes are cathodes, and the second and third electrodes are anodes, and
wherein the first and second electrodes are configured with the third and fourth electrodes so that the first and second electrodes can be controlled and driven independently from the third and fourth electrodes.

5. The medical device of claim 1, wherein the one or more stimulation leads comprise:
a first stimulation lead comprising a first set of electrodes on the first stimulation lead and configured to stimulate the person's vagus nerve; and
a second stimulation lead comprising a second set of electrodes on the second stimulation lead and configured to stimulate at least one selected from among the person's vagus nerve, a branch of the vagus nerve, a heart, a baroreceptor, a renal nerve, a splenic nerve, a carotid sinus nerve, parenchyma tissue, a pancreas, a pancreatic nerve, a spinal cord, a hypoglossal nerve, an ansa cervicalis, a pharyngeal nerve, a cranial nerve, a nerve innervating a liver, and a phrenic nerve.

6. The medical device of claim 5, wherein the second set of electrodes are configured to stimulate the person's vagus nerve or a branch of the vagus nerve, and the first and second stimulation leads are shaped and sized so that the second set of electrodes are positionable farther from the controller than the first set of electrodes are positionable from the controller.

7. The medical device of claim 5, wherein the first and second stimulation leads are separately coupled to the controller.

8. The medical device of claim 5, wherein the first and second stimulation leads are bifurcated from a common lead and are coupled to the controller via the common lead.

9. The medical device of claim 1, wherein the one or more stimulation leads comprise a stimulation lead comprising a first set of electrodes at a first position along the stimulation lead and a second set of electrodes at a second position along the stimulation lead and separated from the first position.

10. The medical device of claim 1, further comprising:
one or more sensors communicatively coupled to the controller and configured to measure one or more biomarkers and/or one or more biophysical properties; and/or
a communication device communicatively coupled to the controller and configured to receive information from an external device.

11. The medical device of claim 10, comprising the one or more sensors, wherein the one or more sensors are configured to be implanted in the person.

12. The medical device of claim 10, comprising the one or more sensors, wherein the one or more sensors are configured to be operated externally from the person.

13. A method of treating obesity in a person, the method comprising:
selecting one configuration of electrodes in a medical device from among at least the following two selectable configurations of electrodes:
a first configuration of electrodes wherein a cathode electrode is more proximal to a head of the person than an anode electrode is to the person's head, and
a second configuration of electrodes wherein a cathode electrode is more distal to the person's head than an anode electrode is to the person's head; and
providing electric stimulation to a vagus nerve of the person utilizing the selected one configuration of electrodes.

14. The method of claim 13, further comprising:
changing the configuration of electrodes from the selected one configuration to an other one configuration of the two selectable configurations of electrodes; and
providing electric stimulation to the vagus nerve utilizing the other one configuration.

15. The method of claim 14, wherein the changing of the configuration of electrodes is in response to a rate of weight loss of the person being below a set threshold rate.

16. The method of claim 14, wherein the changing of the configuration of electrodes is in response to a rate of weight loss of the person being above a set threshold rate.

17. The method of claim 14, wherein the changing of the configuration of electrodes is in response to a change in one or more biomarkers and/or a change in one or more biophysical properties.

18. The method of claim 13, wherein the method further comprises treating one or more medical conditions comorbid with obesity by providing stimulation to at least one selected from among the vagus nerve, a branch of the vagus nerve, a heart, a baroreceptor, a renal nerve, a splenic nerve, a carotid sinus nerve, parenchyma tissue, a pancreas, a pancreatic nerve, a spinal cord, a hypoglossal nerve, an ansa cervicalis, a pharyngeal nerve, a cranial nerve, a nerve innervating a liver, and a phrenic nerve.

19. The method of claim 18, wherein the one or more medical conditions comprise at least one selected from among epilepsy, sleep apnea, obstructive sleep apnea, central sleep apnea, diabetes, hypertension, heart failure, depression, gastroesophageal reflux disease, hypercholesterolemia, pelvic floor disorders, chronic inflammation, auto-immune diseases, and asthma.

20. The method of claim 18, wherein the treating the one or more medical conditions comprises stimulating a portion of the vagus nerve, or a branch of the vagus nerve, different from a portion of the vagus nerve that the electric stimulation is provided to for treating obesity.
